(19) United States
(12) Patent Application Publication
(10) Pub. No.: US 2009/0110606 A1
(43) Pub. Date: Apr. 30, 2009

(54) DISPENSING APPARATUS AND ANALYZER

(75) Inventor: Norichika FUKUSHIMA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/341,444

(22) Filed: Dec. 22, 2008

Related U.S. Application Data
(63) Continuation of application No. PCT/JP2007/059777, filed on May 11, 2007.

(30) Foreign Application Priority Data
Jun. 21, 2006 (JP) 2006-171611

(51) Int. Cl. G01N 35/10; G01N 33/00

(52) U.S. Cl. 422/100; 422/68.1

(57) ABSTRACT

A dispensing apparatus includes a syringe that houses a liquid thereinside; and a plunger that moves forward and backward inside the syringe to thus discharge the liquid from an outlet formed in the syringe to the outside of the syringe. A hydrophilic film is formed on at least either of an inner wall of the syringe and a surface of the plunger.

### CROSS-REFERENCE TO RELATED APPLICATIONS

This application is a continuation of PCT international application Ser. No. PCT/JP2007/059777 filed on May 11, 2007 which designates the United States, incorporated herein by reference, and which claims the benefit of priority from Japanese Patent Application No. 2006-171611, filed on Jun. 21, 2006, incorporated herein by reference.

### BACKGROUND OF THE INVENTION

1. Field of the Invention

The present invention relates to a dispensing apparatus for dispensing a small amount of liquid and an analyzer provided with the dispensing apparatus.

2. Description of the Related Art

In an analyzer for analyzing components of a specimen, such as blood or the like, a technology in which a pressure generated in a syringe is transmitted to a nozzle through a predetermined liquid, and a liquid to be dispensed is dispensed by a predetermined amount from a tip of a nozzle is generally widely employed as a technology for dispensing a specimen and a reagent. In such a dispensing system, air bubbles have sometimes attached to an inner wall of the syringe for housing the liquid or a surface of a plunger for adjusting pressurization and decompression of the syringe, during introduction of the liquid just after the assembly or during repeatedly performed dispensing operations. If a small amount of liquid is dispensed while the air bubbles are thus attached thereto, there have been problems that an amount of the liquid to be dispensed varies to thus cause a decrease in dispense accuracy.

In order to solve the above-mentioned problems, there is conventionally disclosed a technology in which an oscillator is arranged near the tip of the plunger, and the air bubbles attached to the inside of the syringe or the surface of the plunger are removed by ultrasonically vibrating the arranged oscillator (refer to Japanese Patent Application Laid-open No. H11-242040).

### SUMMARY OF THE INVENTION

A dispensing apparatus according to an aspect of the present invention includes a syringe that houses a liquid thereinside; and a plunger that moves forward and backward inside the syringe to thus discharge the liquid from an outlet formed in the syringe to the outside of the syringe. A hydrophilic film is formed on at least either of an inner wall of the syringe and a surface of the plunger.

A dispensing apparatus according to another aspect of the present invention includes a syringe that houses a liquid thereinside; a nozzle that discharges the liquid housed in the syringe to the outside; and a pipe conduit that connects between the syringe and the nozzle, in which a hydrophilic film is formed in at least a part of the area where the liquid moves forward and backward.

An analyzer according to still another aspect of the present invention includes a dispensing apparatus that includes a syringe that houses a liquid thereinside; and a plunger that moves forward and backward inside the syringe to thus discharge the liquid from an outlet formed in the syringe to the outside of the syringe. in the dispensing apparatus, a hydrophilic film being formed on at least either of an inner wall of the syringe and a surface of the plunger.

An analyzer according to still another aspect of the present invention includes a dispensing apparatus that includes a syringe that houses a liquid thereinside; a nozzle that discharges the liquid housed in the syringe to the outside; and a pipe conduit that connects between the syringe and the nozzle, in which a hydrophilic film is formed in at least a part of the area where the liquid moves forward and backward.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a view showing a configuration of a dispensing apparatus in accordance with a first embodiment;

FIG. 2 is an enlarged view of a syringe shown in FIG. 1;

FIG. 3 is a sectional view in which a horizontal plane including an injection axis X1-X1 of the syringe shown in FIG. 2 is a cutting plane;

FIG. 4 is a view showing a configuration of a dispensing apparatus in accordance with a second embodiment;

FIG. 5 is an enlarged view of a syringe shown in FIG. 4;

FIG. 6 is a sectional view in which a horizontal plane including an injection axis X2-X2 of the syringe shown in FIG. 5 is a cutting plane;

FIG. 7 is a view showing a configuration of a dispensing apparatus in accordance with a third embodiment;

FIG. 8 is an enlarged view of a syringe shown in FIG. 7;

FIG. 9 is a view showing a configuration of a dispensing apparatus in accordance with a fourth embodiment;

FIG. 10 is an enlarged view of a tube shown in FIG. 9; and

FIG. 11 is a view showing a configuration of principal parts of an analyzer using the dispensing apparatuses in accordance with the first to fourth embodiments.

### DETAILED DESCRIPTION OF THE PREFERRED EMBODIMENTS

Exemplary embodiments of a dispensing apparatus and an analyzer according to the present invention will be described below with reference to the drawings. The present invention is not limited by these embodiments. Additionally, the same symbol is given to the same component throughout the drawings.

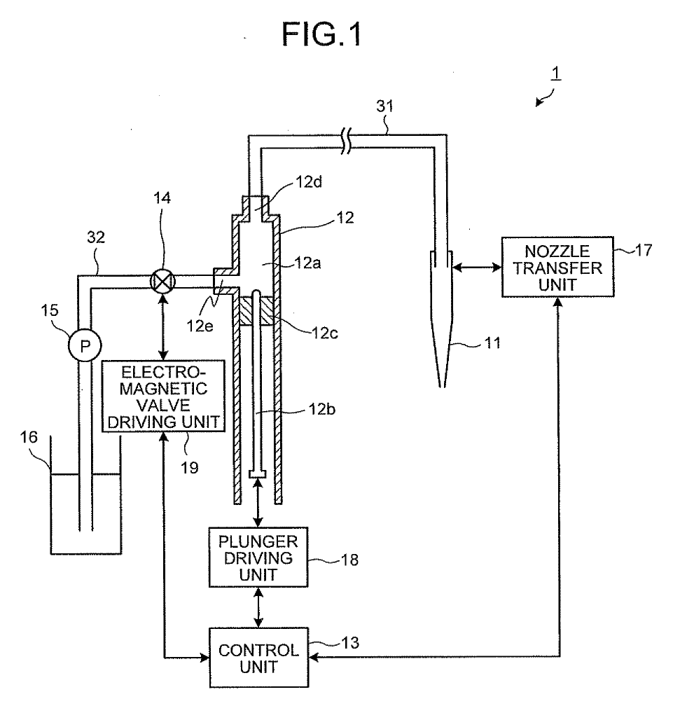

FIG. 1 is an explanatory view schematically showing a configuration of a dispensing apparatus in accordance with a first embodiment of the present invention. A dispensing apparatus 1 shown in FIG. 1 is provided with a hollow nozzle 11 that has a tapered point for sucking and discharging a liquid, a syringe 12 as a pressure generator that is connected to this nozzle 11 through a tube 31 for forming a flow channel of the liquid, and houses the liquid therein to generate pressure for sucking or discharging the liquid from the nozzle 11, and a control unit 13 that controls operations including the suction and discharge of the liquid in the dispensing apparatus 1.

The syringe 12 has a substantially cylindrical shape and has a liquid housing portion 12a for housing a predetermined liquid. A hydrophilic film is formed on an inner wall of the syringe 12, resulting in a configuration that air bubbles are hardly attached to the inner wall of the syringe 12. Further, the syringe 12 has a rod-shaped plunger 12b that adjusts pressure of the liquid housed in the liquid housing portion 12a inside the syringe 12, and a seal member 12c that prevents leakage of the liquid housed in the liquid housing portion 12a and passes through the plunger 12b. Still further, the syringe 12 is provided with an outlet 12d that is connected to the nozzle 11 through the tube 31, and forms a flow channel when the liquid is discharged from the liquid housing portion 12a to the outside of the syringe 12, and an inlet 12e that is provided at a side surface portion of the syringe 12, and forms a flow channel when the liquid is injected into the liquid housing portion 12a from the outside of the syringe 12. The plunger 12b performs forward and backward movements inside the syringe 12 to thus discharge the liquid housed in the liquid housing portion 12a to the outside of the syringe 12 from the outlet 12d formed in this syringe 12. The plunger 12b discharges a liquid of the same volume as the volume that the plunger is advanced inside the syringe 12, to the outside of the syringe 12. Incidentally, the plunger 12b is metal in many cases.

A tube 32 which forms the flow channel when the liquid is injected into the liquid housing portion 12a from the outside of the syringe 12 is connected to the inlet 12e. An electromagnetic valve 14 for controlling a flow of the liquid to be injected, and a pump 15 are sequentially interposed in this tube 32. One end of the tube 32 different from the other end on the syringe 12 reaches a liquid vessel 16 for housing the liquid flowing through the tube 32, thus allowing the liquid for pressure transmission housed in the liquid vessel 16 to be introduced.

The nozzle 11, the plunger 12b, and the electromagnetic valve 14 are connected to the control unit 13 through a nozzle transfer unit 17, a plunger driving unit 18, and an electromagnetic valve driving unit 19, respectively. Among them, the nozzle transfer unit 17 makes the nozzle 11 move in a longitudinal direction and turn about a predetermined axis. In addition, the plunger driving unit 18 makes the plunger 12b move forward and backward. Further, the electromagnetic valve driving unit 19 makes the electromagnetic valve 14 open and close. The control unit 13 which drives and controls these various driving units is achieved by a CPU (Central Processing Unit) or the like having control and calculation functions.

When the liquid to be dispensed is sucked or discharged in the dispensing apparatus 1, the electromagnetic valve 14 is opened to suck the liquid for pressure transmission to be housed in the liquid vessel 16 with the pump 15, and after filling the nozzle 11, the syringe 12, and the tubes 31 and 32 with the liquid for pressure transmission, the electromagnetic valve 14 is closed. Subsequently, when the liquid to be dispensed is sucked or discharged with the nozzle 11, an appropriate suction pressure or discharge pressure is applied to the point of the nozzle 11 through the liquid for pressure transmission by the plunger 12b of the syringe 12 performing the forward and backward movements under the control of the control unit 13. In this case, since an air gap is interposed between the liquid for pressure transmission and the liquid to be dispensed at the point of the nozzle 11, different types of liquids are not mixed with each other.

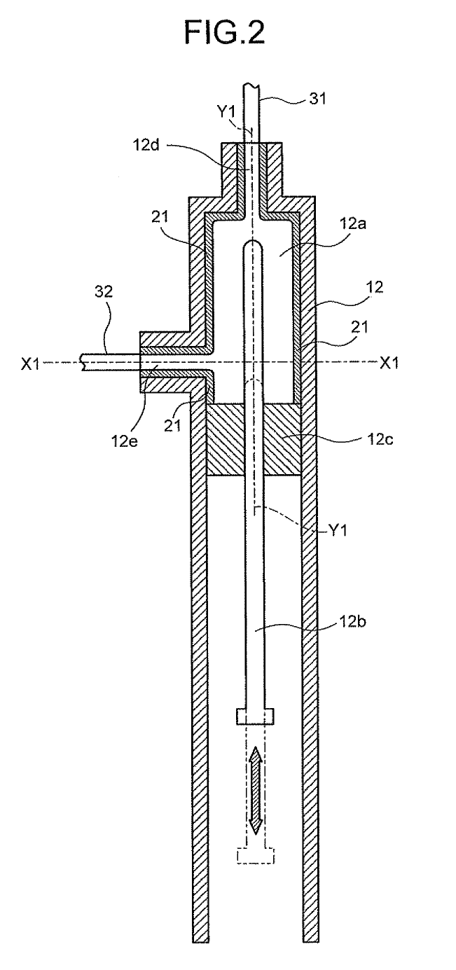

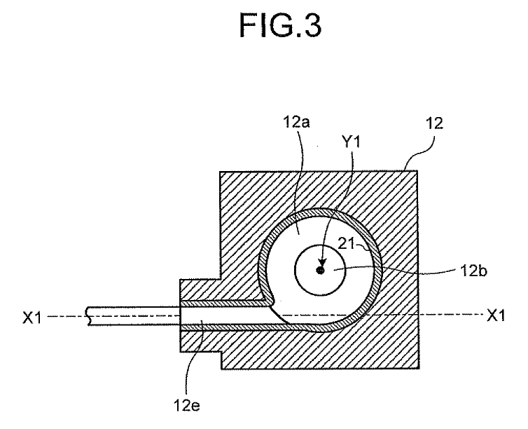

Next, the syringe 12 shown in FIG. 2 will be described with reference to FIG. 2 and FIG. 3. FIG. 2 is a partially enlarged view showing a configuration of the syringe 12. As shown in FIG. 2, the plunger 12b can move forward and backward along with a central axis in a longitudinal direction of the liquid housing portion 12a (in this case, it is coincident with a central axis of the outlet 12d). In FIG. 2, while a state where the plunger 12b is at the most elongated position within the liquid housing portion 12a is represented by a continuous line (hereinafter, it is called the most elongated state), and a state where the plunger 12b is at the most retracted position within the liquid housing portion 12a is represented by a two-point chain line (hereinafter, it is called the most retracted state). FIG. 3 is a sectional view when a horizontal plane in FIG. 2 including an axis X1-X1 shown in FIG. 2 is a cutting plane, and is a sectional view when the plunger 12b is in the most elongated state.

A hydrophilic film 21 which is a thin film with a hydrophilic nature is formed on the inner wall of the syringe 12 as shown in FIG. 2 and FIG. 3. The hydrophilic film 21 is formed of a material having a higher hydrophilic nature than an area of the inner wall of the syringe 12 where the hydrophilic film 21 is not formed. The hydrophilic film 21 is also formed on inner walls which constitute the outlet 12d and the inlet 12e. In other words, the hydrophilic film 21 is formed on the whole area to which the liquid housed in the liquid housing portion 12a contacts among the inner walls of the syringe 12. As a result of this, among the inner walls of the syringe 12, all the areas of the syringe 12 to which the liquid housed in the liquid housing portion 12a contacts are hydrophilized. In the syringe 12, after the hydrophilic film 21 is formed on the inner wall, the tube 31, 32, the seal member 12c, and the plunger 12b are connected thereto.

The hydrophilic film 21 is formed on the inner wall of the syringe 12 using a vapor phase synthetic method. The hydrophilic film 21 is formed as a thin film having a thickness of several angstroms to tens of angstroms composed of a polymeric material, for example, polyvinyl alcohol, phospholipid (Phospholipid polymer) such as 2-Methacryloyloxyethyl Phosphorylcholine, polyethylene glycol, or the like. The vapor phase synthetic method can form a uniform and homogeneous thin film not only on plane shape portions but also on an inner wall of a pipe with a narrow internal diameter. For this reason, the hydrophilic film 21 can be stably formed on all the areas to which the liquid in the inner wall of the syringe 12 contacts by using the vapor phase synthetic method.

Here, if the inner wall of the syringe is hydrophobic, bubbles of the liquid tend to occur during the process where the dry inner wall surface gets wet, and air bubbles generated by the bubbling of the liquid attach to the surface of the inner wall. Moreover, when the inner wall of the syringe is hydrophobic, air bubbles generated by repeatedly moving the plunger forward and backward for the dispensing operation have further attached to the inner wall of the syringe in some cases. As a result of this, the amount of the liquid to be dispensed by the dispensing apparatus cannot be accurately dispensed due to the generation of the air bubbles in the conventional system, so that there has been a problem that dispensing accuracy is decreased.

Meanwhile, in the dispensing apparatus 1 in accordance with the present first embodiment, the hydrophilic film 21 is formed on all the areas of the inner wall of the syringe 12 to which the liquid attaches to thus hydrophilize the inner wall of the syringe 12. The air bubbles are hardly attached to a hydrophilic region as compared with a hydrophobic region. For this reason, when the inner wall of the syringe has a hydrophilic nature as the dispensing apparatus 1, bubbling of the liquid hardly occurs in the processes where the dry inner wall plane gets wet, and generation of the air bubbles is low. Further, in the dispensing apparatus 1, even when air bubbles are generated during the dispensing operation, the air bubbles do not attach to the inner wall of the syringe, thus allowing the air bubbles to be also discharged from the inside of the syringe smoothly. Still further, in the dispensing apparatus 1, since it is not necessary to provide the vibration mechanism that vibrates the plunger for removing the air bubbles as the dispensing apparatus in accordance with the conventional system, a simple configuration can be achieved. Yet still further, in the dispensing apparatus 1, since the plunger 12b passes through the inside of the seal member 12c, the plunger 12b and the area on which the hydrophilic film 21 is formed in the inner wall of the syringe 12 do not contact with each other. As a result, in the dispensing apparatus 1, even when the forward and backward movements of the plunger 12b are performed, the hydrophilic film 21 formed on the inner wall of the syringe 12 is hardly peeled off.

As described above, according to the present first embodiment, it is possible to provide the dispensing apparatus that can easily remove the air bubbles attached to the syringe and also has a simple configuration, thereby allowing the manufacturing cost in the dispensing apparatus to be suppressed low.

Note herein that, although the case where the hydrophilic film 21 is formed using the vapor phase synthetic method has been described in the present first embodiment, it is not limited to this. The hydrophilic film 21 may be formed using a wet method in which a solvent including a thin film material is used. The hydrophilic film having a uniform film thickness can be formed on the inner wall of the syringe also when the wet method is used. Additionally, the hydrophilic film 21 may be formed using a sol-gel process in which a sol thin film material is dried after being coated on the inner wall of the syringe 12. The thin film can be formed on a cylindrically-shaped inner wall like the syringe also when the sol-gel process is employed. Meanwhile, when forming the hydrophilic film only in a desired area using the vapor phase synthetic method, the wet method, and the sol-gel process, the area is selected by masking an area where a non-hydrophilic film is formed with covering or an syringe opening with plug setting to form the hydrophilic film 21.

In addition, the liquid for pressure transmission housed in the liquid vessel 16 is an incompressible fluid, such as ion exchange water, distilled water, de-aired water, or buffer solution. Such a liquid can be not only used for dispensing the liquid to be dispensed, but also utilized as a cleaning liquid for cleaning the inside of the nozzle 11 and for cleaning other vessels. Moreover, it is also possible to house the liquid to be dispensed in the liquid vessel 16 instead of the liquid for pressure transmission, and to dispense the liquid to be dispensed as it is.

Meanwhile, in order to remove the generated air bubbles, the plunger 12b is stopped, and the electromagnetic valve 14 is opened under the control of the control unit 13, so that the liquid is injected into the liquid housing portion 12a by predetermined pressure from the inlet 12e. The liquid injected into the liquid housing portion 12a inside the syringe 12 from the inlet 12e flows toward the outlet 12d so as to swirl around the plunger 12b. Configuring the syringe 12 as shown in FIG. 3 can generate a swirl flow to thus remove the air bubbles attached to the inner wall of the syringe 12 or the surface of the plunger 12b by the swirl flow.

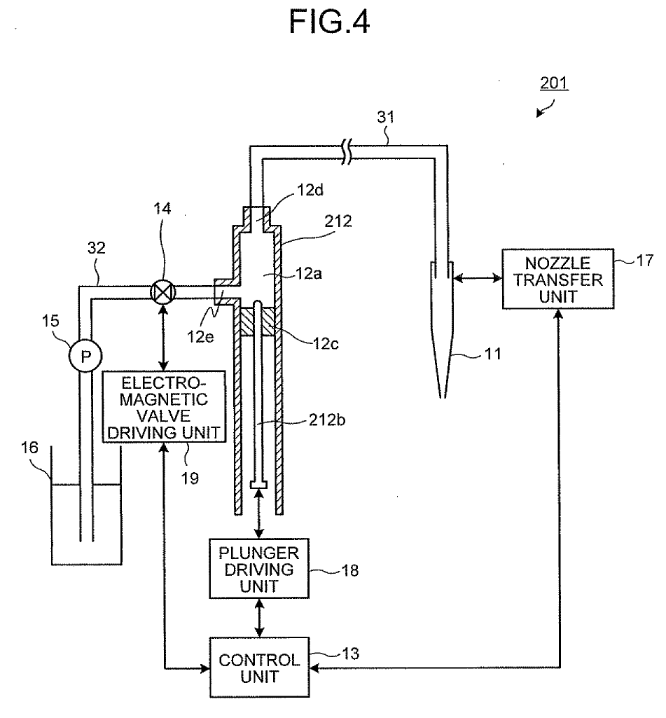

Next, a second embodiment will be described. FIG. 4 is an explanatory view schematically showing a configuration of a dispensing apparatus in accordance with the present second embodiment. As shown in FIG. 4, a dispensing apparatus 201 in accordance with the second embodiment is provided with a syringe 212 having a plunger 212b in place of the plunger 12b in the dispensing apparatus 1 shown in FIG. 1.

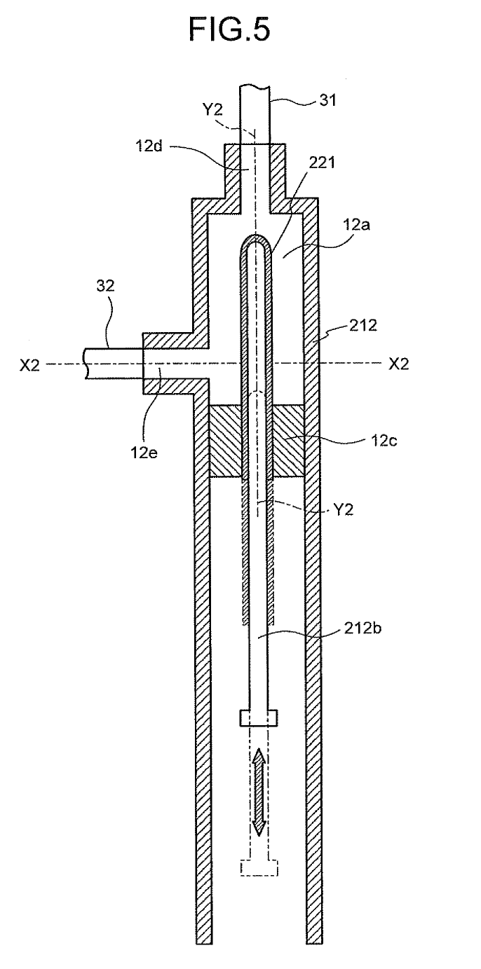

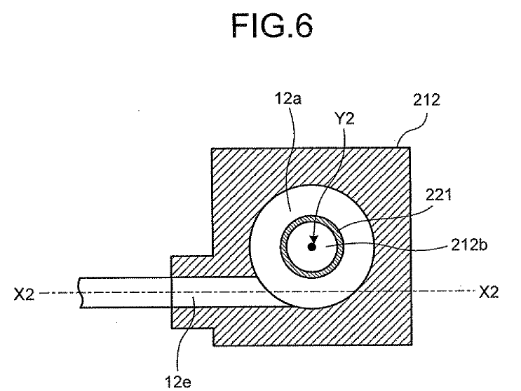

The syringe 212 shown in FIG. 4 will be described with reference to FIG. 5 and FIG. 6. FIG. 5 is a partially enlarged view showing a configuration of the syringe 212. FIG. 6 is a sectional view when a horizontal plane in FIG. 5 including an axis X2-X2 shown in FIG. 5 is a cutting plane, and it is a sectional view when the plunger 212b is in the most elongated state.

As shown in FIG. 5 and FIG. 6, a hydrophilic film 221 is formed on a surface of the metal plunger 212b. The hydrophilic film 221 has a higher hydrophilic nature than the surface of the plunger 212b where the hydrophilic film 221 is not formed. Among the surfaces of the plunger 212b, the hydrophilic film 221 is formed in an area where the plunger 212b contacts to the liquid housed in the liquid housing portion 12a in the most elongated state into the inside of the syringe 212, and an area where the plunger 212b and the seal member 12c contact to each other during forward and backward movements of the plunger 212b. For this reason, the air bubbles are hardly attached to the surface of the plunger 212b. It is to be noted that the hydrophilic film is not formed on an inner wall of the syringe 212 as shown in FIG. 5 and FIG. 6 as compared with FIG. 2 and FIG. 3.

This hydrophilic film 221 is a ceramic film, and it is formed on the surface of the plunger 212b using a vacuum heating method in which the film is formed in vacuum where a metal chloride and other source gases, such as H₂, CH₄, NH₃, CO₂, or the like, which are used as a material of a thin film, are supplied, by heating them at a processing temperature of 800 to 1500 degrees C. Using this vacuum heating method makes it possible to form the hydrophilic film 221 having high wear and abrasion resistance on a sliding surface of the plunger 212b to the seal member 12c in the syringe 212. As the ceramic film composing the hydrophilic film 221, TiN, BrC₄, DLC, and the like, which are subjected to vacuum heating processing after film forming are included. The hydrophilic film 221 formed on the plunger 212b has a film thickness of TiO₂, for example.

As described above, since the ceramic film is formed as the hydrophilic film 221, the surface of the plunger 212b has wear and abrasion resistance while having a hydrophilic nature. The area where the hydrophilic film 221 is formed includes the area where the plunger 212b contacts to the liquid in the most elongated state of the plunger 212b, and the sliding surface which is the surface of the plunger 212b of a portion where the plunger 212b and the seal member 12c contact to each other during the forward and backward movements of the plunger 212b. Since the hydrophilic film 221 having wear and abrasion resistance is thus formed on the sliding surface, the hydrophilic film 221 is hardly peeled off even when the plunger 212b repeatedly passes through the inside of the seal member 12c for the dispensing operation. Further, a temporal change in a sliding resistance between the plunger 212b and the seal member 12c is small as compared with a case where the hydrophilic film 221 is not formed.

As described above, in the dispensing apparatus 201 in accordance with the present second embodiment, the hydrophilic film 221 is formed on the surface of the plunger 212b to thus prevent adhesion of the air bubbles to the surface of the plunger 212b. Hence, according to the dispensing apparatus 201, it is possible to prevent generation of the air bubbles inside the syringe 212, and to also discharge the air bubbles from the inside of the syringe 212 smoothly. Additionally, in the dispensing apparatus 201, since it is not necessary to provide a vibration mechanism which vibrates the plunger for removing the air bubbles, a simple configuration can be achieved. Moreover, in the dispensing apparatus 201, since the hydrophilic film 221 having high wear and abrasion resistance is formed on the surface of the plunger 212b, the temporal change in the sliding resistance of the plunger 212b is reduced, thereby allowing the dispensing accuracy in the dispensing apparatus 201 to be maintained. Thus, according to the present second embodiment, a similar effect to that of the first embodiment can be achieved.

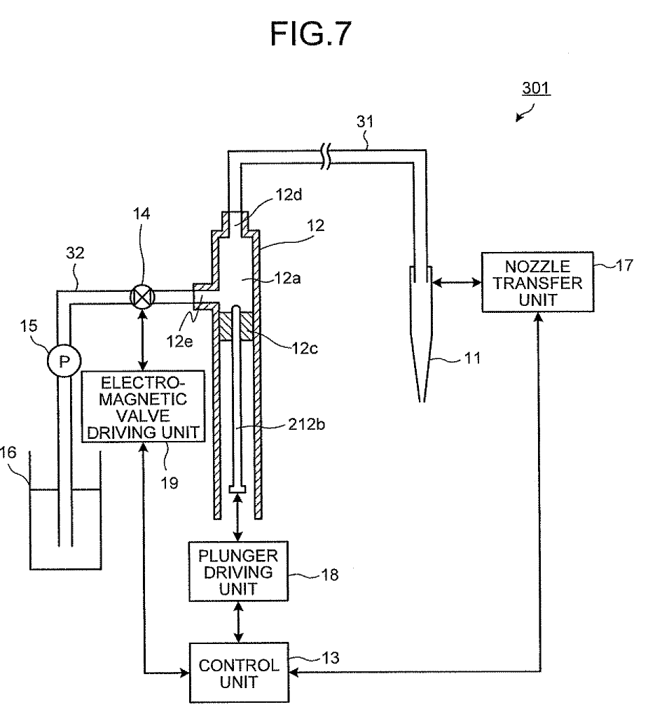

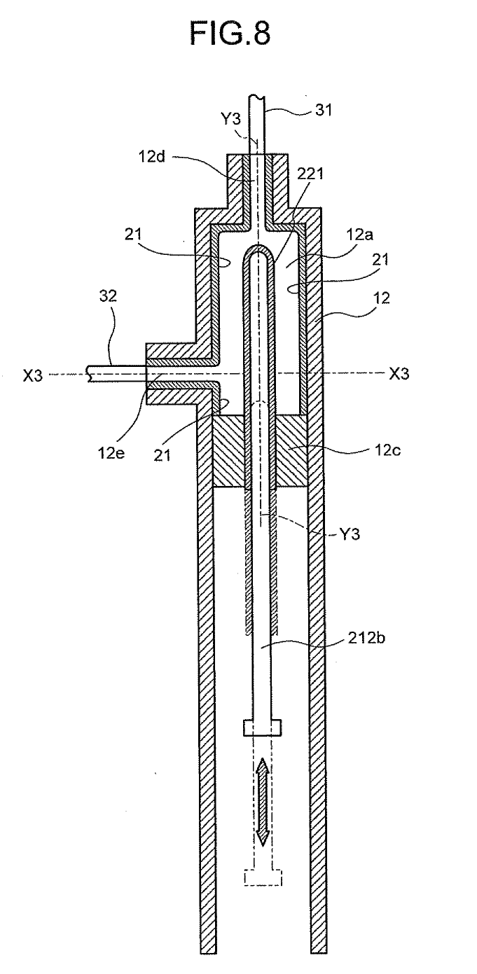

Next, a third embodiment will be described. FIG. 7 is an explanatory view schematically showing a configuration of a dispensing apparatus in accordance with the present third embodiment. Meanwhile, FIG. 8 is a partially enlarged view showing a configuration of the syringe 12 shown in FIG. 7. As shown in FIG. 7 and FIG. 8, the dispensing apparatus 301 in accordance with the third embodiment has a configuration provided with the syringe 12 described in the first embodiment, and the plunger 212b described in the second embodiment. The hydrophilic films 21 and 221 are formed on the inner wall of the syringe 12 and the surface of the plunger 212b.

As described above, according to the third embodiment, forming the hydrophilic films 21 and 221 on both of the inner wall of the syringe 12 and the surface of the plunger 212b makes it possible to achieve the dispensing apparatus that has a simple configuration and reliably prevents occurrence of the air bubbles and adhesion of the air bubbles.

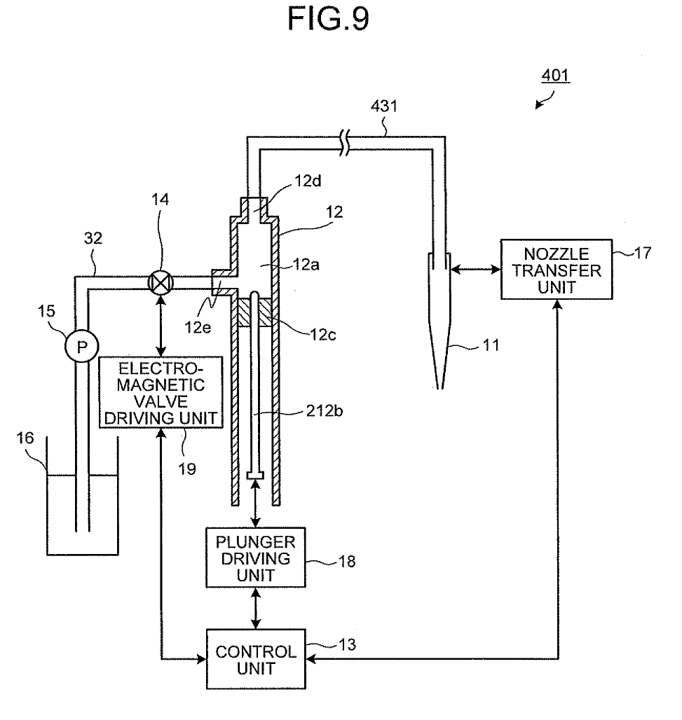

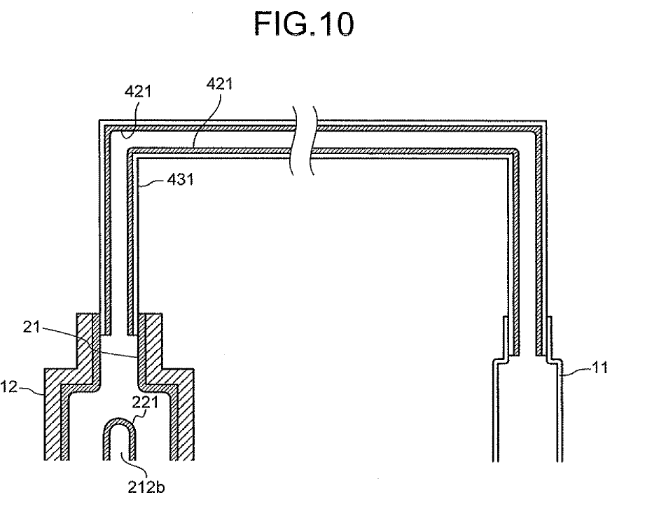

Next, a fourth embodiment will be described. FIG. 9 is an explanatory view schematically showing a configuration of a dispensing apparatus in accordance with the present fourth embodiment. Meanwhile, FIG. 10 is a partially enlarged view showing a configuration of a tube 431 shown in FIG. 9. As shown in FIG. 9 and FIG. 10, a dispensing apparatus 401 in accordance with the fourth embodiment is provided with the tube 431 in place of the tube 31 shown in FIG. 7.

A hydrophilic film 421 which is a thin film having a hydrophilic nature is formed on an inner wall of the tubular tube 431 as shown in FIG. 10. The hydrophilic film 421 is formed of a material with a higher hydrophilic nature than the area where the hydrophilic film 421 is not formed. The hydrophilic film 421 is formed in the whole area within the tube 431, and all the areas to which the liquid discharged from the syringe 12 contacts are hydrophilized. Additionally, the hydrophilic film 421 is formed as a thin film having a thickness of several angstroms to tens of angstroms composed of a polymer, for example, polyvinyl alcohol, phospholipid (Phospholipid polymer) such as 2-Methacryloyloxyethyl Phosphorylcholine, polyethylene glycol, or the like, using the vapor phase synthetic method, the wet method, or the sol-gel process in a manner similar to that of the hydrophilic film 21 in the first embodiment.

As described above, according to the fourth embodiment, the hydrophilic film 421 is formed also on the inner wall of the tube 431 where the liquid discharged from the syringe 12 moves forward and backward, in addition to the inner wall of the syringe 12 and the surface of the plunger 212b, thereby allowing the dispensing apparatus that has a simple configuration and reliably prevents occurrence of the air bubbles and adhesion of the air bubbles to be achieved.

It is to be noted that the case where the hydrophilic films 21, 221, and 421 are formed on all the inner wall of the syringe 12, the surface of the plunger 212b, and the inner wall of the tube 431 has been described as the fourth embodiment, but without being limited to this, the hydrophilic films 21, 221, and 421 may be formed on a part of the inner wall of the syringe 12, the surface of the plunger 212b, and the inner wall of the tube 431. The hydrophilic films 21, 221, and 421 are formed on at least a part of the inner wall of the syringe 12 which is the area where the liquid moves forward and backward, the surface of the plunger 212b, and the inner wall of the tube 431, thereby allowing occurrence of the air bubbles and adhesion of the air bubbles to be further prevented than the conventional system.

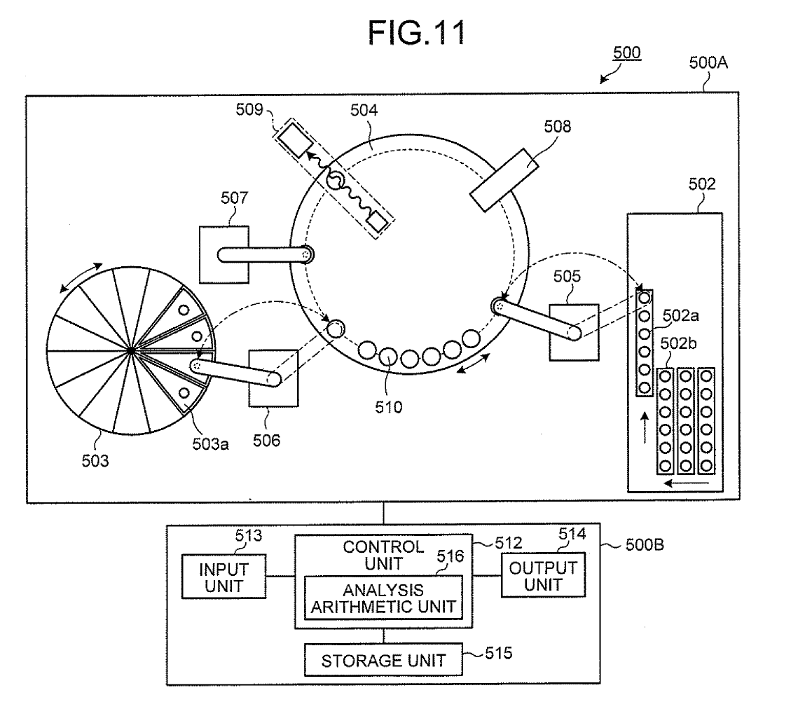

Moreover, the dispensing apparatuses 1, 201, 301, and 401 in accordance with present first to fourth embodiments are applicable to an analyzer that analyzes components of a specimen. FIG. 11 is an explanatory view showing a configuration of principal parts of an analyzer provided with the dispensing apparatuses 1, 201, 301, and 401. An analyzer 500 shown in FIG. 11 has a measuring system 500A that dispenses to a reaction vessel a specimen and a reagent which are samples, respectively, to optically measure reactions caused within the reaction vessel, and a control analysis system 500B that drives and controls this measuring system 500A and also analyzes measurement results in the measuring system 500A, wherein a biochemical or immunological analysis of the components of a plurality of specimens is conducted automatically and continuously while these two systems are cooperating with each other.

The measuring system 500A of the analyzer 500 is provided with a specimen transfer unit 502 that houses a plurality of racks 502b in which a specimen vessel 502a for housing specimens, such as blood, body fluids, or the like is mounted, to sequentially transfer them, a reagent table 503 that holds a reagent vessel 503a, and a reaction table 504 that holds a reaction vessel 510 for reacting a specimen with a reagent. Further, the measuring system 500A is provided with a specimen dispensing unit 505 that dispenses the specimen housed in the specimen vessel 502a on the specimen transfer unit 502 to the reaction vessel 510, a reagent dispensing unit 506 that dispenses the reagent housed in the reagent vessel 503a on the reagent table 503 to the reaction vessel 510, a stirring unit 507 that stirs a liquid dispensed inside the reaction vessel 510, a cleaning unit 508 that cleans the reaction vessel 510, and a light measuring unit 509 that receives and measures intensity for every component of a light which was irradiated from a predetermined light source and passed through the inside of the reaction vessel 510, by a photodiode or a photomultiplier.

The reagent table 503 and the reaction table 504 are rotatable freely on a horizontal plane around a plumb line passing through the center of each table as an axis of rotation by driving a stepping motor under control by the control analysis system 500B. While an openable and closable cover is provided at an upper part of each table, a constant temperature bath is provided at a lower part of each table, and thus evaporation or deterioration of the specimen and the reagent in various vessels is suppressed by maintaining the reagent vessel 503a and the reaction vessel 510 in a constant temperature state.

The dispensing apparatuses 1, 201, 301, and 401 in accordance with the first to fourth embodiments can be applied to the specimen dispensing unit 505 and the reagent dispensing unit 506.

Next, a configuration of the control analysis system 500B of the analyzer 500 will be described. The control analysis system 500B controls the analyzer 500, and it is also provided with a control unit 512 that carries out an operation to analyze the measurement results in the measuring system 500A, an input unit 513 that receives inputs of information required for the analysis of the specimen and an operation instruction signal of the analyzer 500, an output unit 514 that outputs information including the results of the analysis, and a storage unit 515 that stores the information including the results of the analysis.

The control unit 512 has an analysis arithmetic unit 516 that carries out an analysis operation of the components of the specimen based on the measurement results in the measuring system 500A. This control unit 512 carries out control of various operations, the analysis operation, and the like of the analyzer 500 by reading a program stored in the storage unit 515 from a memory thereof. For this reason, the control unit 512 may also have functions of the control unit 13 of the dispensing apparatuses 1, 201, 301, and 401 which are applied as the specimen dispensing unit 505, the reagent dispensing unit 506, and the cleaning unit 508.

When the control analysis system 500B having the configuration described above receives the measurement result from the light measuring unit 509, the analysis arithmetic unit 516 reads the analysis information of the specimen to be measured from the storage unit 515, and carried out the analysis operation of the measurement results. In this analysis operation, absorbance of the reaction solution is calculated based on the measurement results sent from the light measuring unit 509, and a calibration curve obtained from a standard sample or analytical parameters included in the analysis information are utilized in addition to the calculation results to thus calculate the components of the reaction solution or the like quantitatively. While the results of the analysis thus obtained are outputted from the output unit 514, they are stored and memorized in the storage unit 515.

Since the analyzer 500 can dispense highly accurately the reagent and specimen with a predetermined amount by being provided with the dispensing apparatuses 1, 201, 301, and 401 that can prevent occurrence of the air bubbles and adhesion of the air bubbles, it is possible to achieve improvement in analysis accuracy.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A dispensing apparatus, comprising:
a syringe that houses a liquid thereinside; and
a plunger that moves forward and backward inside the syringe to thus discharge the liquid from an outlet formed in the syringe to the outside of the syringe, a hydrophilic film being formed on at least either of an inner wall of the syringe and a surface of the plunger.

2. The dispensing apparatus according to claim 1, wherein the hydrophilic film formed on the surface of the plunger is a ceramic film.

3. The dispensing apparatus according to claim 2, wherein the ceramic film is formed on a sliding surface which is the surface of the plunger where the plunger and the syringe contact to each other during forward and backward movements of the plunger.

4. The dispensing apparatus according to claim 1, wherein the plunger discharges a liquid of the same volume as the volume that the plunger is elongated inside the syringe, to the outside.

5. The dispensing apparatus according to claim 1, wherein the hydrophilic film is formed using a vapor phase synthetic method.

6. A dispensing apparatus, comprising:
a syringe that houses a liquid thereinside;
a nozzle that discharges the liquid housed in the syringe to the outside; and
a pipe conduit that connects between the syringe and the nozzle, in which a hydrophilic film is formed in at least a part of the area where the liquid moves forward and backward.

7. The dispensing apparatus according to claim 6, wherein the hydrophilic film is formed on an inner wall of the pipe conduit.

8. The dispensing apparatus according to claim 6, wherein the hydrophilic film is formed using a vapor phase synthetic method.

9. An analyzer comprising a dispensing apparatus, the dispensing apparatus including:
a syringe that houses a liquid thereinside; and
a plunger that moves forward and backward inside the syringe to thus discharge the liquid from an outlet formed in the syringe to the outside of the syringe, a hydrophilic film being formed on at least either of an inner wall of the syringe and a surface of the plunger.

10. An analyzer comprising a dispensing apparatus, the dispensing apparatus including:
a syringe that houses a liquid thereinside;
a nozzle that discharges the liquid housed in the syringe to the outside; and
a pipe conduit that connects between the syringe and the nozzle, in which a hydrophilic film is formed in at least a part of the area where the liquid moves forward and backward.
